(19) United States
(12) Patent Application Publication
(10) Pub. No.: US 2014/0378919 A1
(43) Pub. Date: Dec. 25, 2014

(54) Easily Washable and Drainable Pouch

(71) Applicant: JEZEKIEL BEN-ARIE, CHICAGO, IL (US)

(72) Inventor: JEZEKIEL BEN-ARIE, CHICAGO, IL (US)

(21) Appl. No.: 14/304,909

(22) Filed: Jun. 14, 2014

Related U.S. Application Data
(60) Provisional application No. 61/838,230, filed on Jun. 22, 2013.

(51) Int. Cl. A61F 5/442

(52) U.S. Cl. CPC A61F 5/442
USPC 604/334

(57) ABSTRACT

A washable pouch which enables accumulating solid waste and also to drain and wash it when the pouch is filled. The washable pouch has an abdominal inlet, a draining outlet and a washing inlet. The washable pouch has a flat structure constructed of two planar sheets of flexible material, which are fused together at their margins all along their perimeter except for inlet and outlet openings. Both washing inlet and draining outlet have funnel shapes with seals which can block all flows when closed. The washing inlet which is installed at the pouch's top can be used to expel gasses and also to have an air filter. The washing inlet's funnel shape facilitates pouring water into the pouch, thus liquefying the solid waste and enabling easier draining. The abdominal inlet is surrounded by an adhesive layer which has tapered boundary thereby reducing abdominal skin irritation.

### CROSS-REFERENCE TO RELATED APPLICATIONS

This application claims the benefit of a provisional patent application:

Ser. No. 61/838230 filed on 22 Jun. 2013

### FEDERALLY SPONSORED RESEARCH

Not Applicable

### SEQUENCE LISTING OR PROGRAM

Not Applicable

### BACKGROUND OF THE INVENTION

1. Field of the Invention

The invention is in the area of means for improving the process of draining and washing ostomy pouches.

2. Description of Related Art and Design Considerations

Drainable pouches are being used by people who underwent ileostomy also referred to as ostomy. Ostomy is the construction of an artificial opening from the ileum through the abdominal wall, permitting drainage of the contents of the small intestine. The drainable pouch is attached to the abdominal opening and collects the solid waste drained from the small intestine. Every few hours the pouch is filled and needs to be drained. Usually these pouches have the shape of a bag and are made of flexible material such as plastic sheets. In addition to the pouch's inlet, which is attached to the abdominal opening (also called stoma), prevalent pouches have an outlet from which the pouch is being drained. This outlet is sealed during accumulation and can be opened for drainage and re-sealed afterwards.

When the user drains the pouch, the user has to open the outlet and to squeeze the pouch in order to drain it. The inventor personally had ileostomy and had been using these pouches for few years. Users of ostomy pouches are experiencing a major difficulty in the draining process. Since the solid waste is highly viscous and sticky, it adheres to the inner walls of the pouch and draining it requires a lot of squeezing. Even after a lot of squeezing the pouch is not completely emptied and remains with considerable residue inside. To alleviate this problem, the inventor developed a washing and draining procedure in which the user is adding water to the pouch through the draining outlet. Next, the user mixes the solid waste with the water into a fluent mixture and then drains the mixture via the outlet. This process needs repeating several times until the pouch is entirely cleaned. It was found that this washing procedure is faster, easier, more hygienic and achieves much better drainage.

The inventor thinks that the washing procedure can be significantly improved if the pouch had also an additional washing inlet into which the user could add water to the pouch and then drain the mixture which becomes more fluent, via the draining outlet. The reason for a need for an additional washing inlet is because the draining outlet is pointing downwards and is located at the bottom of the pouch. This arrangement complicates and hinders the washing procedure since the user has to bend the outlet upwards, open it while making sure that the solid waste remains in the pouch when the water is filled. Next, the user has to close the outlet and keep it closed while mixing the solid waste with the water and only then the user can re-open the outlet and drain the mixture. This washing procedure has to be repeated few times if one wants an entirely empty pouch.

However, if the pouch could have an additional inlet, i.e. a washing inlet installed at the top of the pouch (in addition to the abdominal inlet which is attached to the abdominal opening), then the washing procedure could be significantly simplified and eased. Pouring water into the washing inlet on top rinses washes and drains the solid waste more efficiently downwards through the drainage outlet. To facilitate water input the washing inlet could have a shape of a funnel, which enables to pour water into the pouch from a variety of water vessels. It also has to have a seal to prevent accidental leakage from the pouch. The washing inlet could also enable easy expulsion of gases usually excreted from the solid waste and accumulated in the pouch. To get reed of the gasses all the user needs to do is to momentarily open the washing inlet seal and let the gasses evaporate.

Installing a washing inlet could have additional advantages. For example, the manufacturer could install on the inlet's wall an air filter that enables to remove excess gasses discharged from the solid waste. Usually such filters are installed at the upper side of the pouch but they become clogged with solid waste even after a very short use. Such clogging effectively blocks the filter and renders it useless. Installing a filter on the inlet wall separates it more effectively from the solid waste in the pouch because the washing inlet extends from the very top of the pouch.

Another advantage of the washable pouch described in this application is its very flat structure and relatively cheap to manufacture. The pouch including the washing inlet and the draining outlet is manufactured by fusing two flat sheets of flexible material. This flat structure is not conspicuous when the user wears the pouch under the user's clothes. Other inventions described below which employ various configurations of pipes are much more bulky and expensive in manufacturing.

Other problems that are routinely encountered by users who use the pouches are skin wounds and irritations that result from the sharp edges of the adhesive layer which adheres the abdominal inlet to the abdominal skin around the abdominal opening. For some reason, this adhesive layer, which comprises of a disc made of adhesive material, has sharp edges on its circumference. The user could have avoided many irritations and wounds if these sharp edges would have been tapered or rounded.

3. Prior Art

Searching the PTO database for inventions which employ various means for washing pouches yielded several patents and patent applications. But all of them were entirely dissimilar to our invention. Several patent applications propose various methods for flushing ostomy pouches. In US 20080060679 Rosenberg proposes to use a special flushing water container which connects to the pouch's outlet. Rosenberg claims no water spills but since the outlet is located at the bottom of the pouch, it presents a major problem how to disconnect the container without spilling.

In U.S. Pat. No. 4,692,159 Kuzemchak invents a flushing nozzle which fits the pouch's inlet and which connects to a water pipe. However, it seems that Kuzemchak forgot that this inlet is connected to the user's abdominal opening and removing the pouch when it is filled with solid waste is almost impossible without spilling. Very similar approaches are presented in U.S. Pat. No. 6,224,581 by Withers et al. and in US 20060155152 by Walker.

In U.S. Pat. No. 6,582,410 Rutman invents an ostomy drainage apparatus which consists of a tube which connects the pouch's outlet to a large container. We fail to see how this is better than simply draining the pouch directly to a toilet bowl.

In U.S. Pat. No. 4,941,878 Petrik invented an ostomy pouch flasher which is basically a metal pipe with few holes at its end and a water pipe inlet at its other end. This pipe is designed to be inserted into the pouch and help flushing it.

In US 2006/0106354 A1 Vantrustenberge invents an ostomy pouch with 3 openings. The first one serves as an inlet which is connected to the user's abdominal opening. The second one is a wide pouch outlet which is used for pouch draining. The third opening in the pouch is a port connected to a one way valve which has a stem pipe at the valve's outlet. The third opening has two uses. The first use is for expelling gases accumulated in the pouch wherein the valve is activated by pressing the pouch and creating enough pressure which opens the one directional valve and allowing the gas to exit. The second use of the third opening is to use it for flushing the bag after draining. The stem pipe is connected to a water source and water is being forced into the pouch. This invention has two major flaws. The first one is the need for a pressurized water source that needs to be connected to the stem pipe for flushing. The second problem is a contradiction in the invention design because if the one way valve is designed to expell gasses from the pouch outwards it will block any flow of rinsing water inwards into the pouch. A similar invention is presented by King in U.S. Pat. No. 6,918,898 B2. King uses an enema bag as an irrigation water source which is connected by a tube to a plapped opening in the pouch. In our opinion, enema's dripping is much too slow for pouch rinsing and a flapped input will allow likage of gasses and waste from the pouch.

Our invention is entirely different from Vantrustenberge's because our invention does not have a valve or a pipe input. Our invention has a wide funnel inlet which allows to rinse the pouch from any water vessel, bottle or pitcher. Our inlet has also a seal which enables to seal the inlet when not in use. Expulsion of excess gases from the pouch can also be achieved simply by momentarily opening the inlet seal and sealing it afterwards.

Schena in U.S. Pat. No. 8,016,802 B1 invented a pouch irrigator which consists of a curved porous pipe installed inside the pouch. The irrigator has a pipe inlet installed at a pouch's opening. This invention is not practical because it requires a pressurized water source connected to the irrigator's inlet. It is also very expensive solution for a disposable pouch. A similar pouch irrigator composed of a porous pipe installed inside the pouch was invented by Fenton in U.S. Pat. No. 4,654,037. Similar invention with pipe irrigation is in U.S. Pat. No. 7,918,836 B2 by Gill et al.

An ostomy suction system was invented by James in U.S. Pat. No. 8,167,857 B2, which uses vacuum tube connected to the pouch outlet for efficient draining. Fundock in U.S. Pat. No. 5,470,325 invented an irrigation and venting assembly which is composed of a simple pipe inlet installed on the top of the pouch. The pipe inlet needs to be connected to a water source for rinsing.

An ostomy bag cleaning apparatus is described in U.S. Pat. No. 5,503,633 by Saunders. The apparatus is actually a drainage construction which enables to flush used ostomy pouches with a water sprinkler over the toilet. Similar approach to pouch cleaning is in US 2006/0237039 A1 by Sarvis. An peelable ostomy bag which can be cleaned by peeling it from the abdominal opening is invented by Pedersen et al. in US 2009/0163885 A1.

In summary, our invention is entirely different from all the inventions described above because our invention does not have a valve or a pipe input. Our invention is the only one which has a wide funnel inlet which allows to rinse the pouch from any water vessel, bottle or pitcher. Our inlet has also a seal which enables to seal the inlet when not in use. Expulsion of excess gases from the pouch can also be achieved simply by momentarily opening the inlet seal and sealing it afterwards. In addition our invention presents the only solution to an inlet and a pouch with flat structure which is not bulky and quite cheap to manufacture.

### BRIEF SUMMARY OF THE INVENTION

The present invention pertains to a novel pouch structure which enables easy washing and draining of ostomy pouches. Usually, ostomy pouches are made of flexible material such as plastic and have an inlet opening which is attached to the abdominal opening of the user. The pouch accumulates the solid waste discharged from the abdominal opening. Every few hours the pouch is filled and needs to be drained. Usually pouches have an outlet from which the pouch is being drained. This outlet is sealed most of the time when solid waste is being accumulated and can be opened for drainage and re-sealed afterwards. When the user drains the pouch, the user has to open the outlet and to squeeze the pouch in order to force out the solid waste. Users are experiencing a major difficulty in such draining process since the solid waste is highly viscous and sticks to the pouch walls. Large portions of the solid waste still remain in the pouch even after a lot of squeezing. Few inventions (described in the previous section) propose to improve the drainage by mixing the solid waste with water, which converts it to more fluent mixture that can be easily drained. These inventions propose to add water to the pouch via various structures of pipe inlets installed in the pouch. There are two major problems with these approaches which renders them impractical. Firstly, pipe inlets need to be connected to a water source via a flexible pipe. This requirement limits the usage of such pouches only to special toilets with a water source and fitting pipes. Since most toilets are not equipped with such water sources, users of pouches with pipe inlet can use them only in few locations. Even more serious problem arises from the requirement to include in each pouch an inner pipe with a pipe inlet. Adding a pipe structure to each pouch makes it quite bulky and expensive since ostomy pouches need to be replaced with new ones every few days.

The present invention proposes an economical and efficient solution to draining and washing pouches by adding an additional washing inlet at the top of the pouch. This solution is quite economical because this inlet is made of the same flexible material as the rest of the pouch and has the same sealing arrangement as the pouch's outlet. The invention also enables efficient drainage and washing because it does not require any special water source and the inlet, which has a funnel shape can be filled from a variety of water vessels and containers. Since the proposed inlet is located at the top of the pouch and the drainage outlet is located at the bottom of the pouch, the washing and rinsing process is quite efficient. To facilitate water input, the washing inlet has a shape of a funnel, It is also equipped with a seal to prevent accidental leakage from the inlet.

The washing inlet could also enable easy expulsion of gases usually excreted from the solid waste and accumulated in the upper part of the pouch. To get reed of these gasses all the user needs to do is to momentarily open the washing inlet seal and let the gasses evaporate.

Another advantage of the washable pouch described in this application is its very flat structure which is relatively cheap to manufacture. The pouch including the washing inlet, the abdominal inlet and the draining outlet is manufactured by fusing two flat sheets of flexible material at their margins. This flat structure is not conspicuous when the user wears the pouch under the user's clothes. Other inventions which employ various configurations of pipes are much more bulky and expensive in manufacturing.

### BRIEF DESCRIPTION OF THE DRAWINGS

FIG. 1 describes the rear view of an embodiment of the washable pouch 1A, in which the rear flexible sheet 1F with the adhesive abdominal adhesive disk 2B is shown.

FIG. 2 illustrates the front view of an embodiment of the washable pouch 1A in which the front flexible sheet 1E is shown.

FIG. 3 shows two side 3D views of embodiments of the washable pouch.

FIGS. 4-8 describe a proposed washing and draining procedure of the pouch.

### DETAILED DESCRIPTION OF THE INVENTION AND DRAWINGS

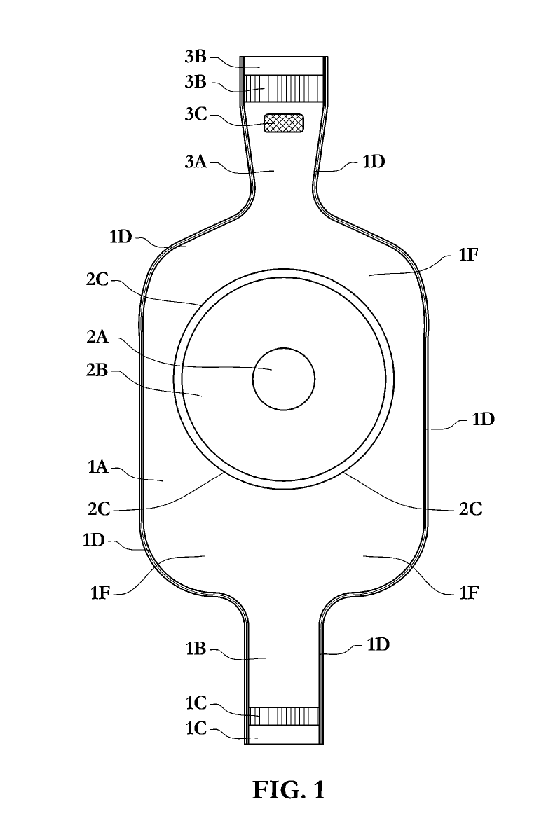

FIG. 1 describes a rear view of an embodiment of the washable pouch 1A. The rear flexible sheet 1F is illustrated with the abdominal inlet 2A and the adhesive disk 2B which surrounds the abdominal inlet 2A. The adhesive disk 2B is installed on the rear flexible sheet 1E. During usage, the pouch is attached to the abdominal skin by the adhesive disc 2B which adheres to the abdominal skin around the abdominal opening (stoma). At the bottom of pouch 1A is an outlet 1B for draining the accumulated solid waste. The draining outlet 1B can be sealed by the seal 1C. The pouch has also a washing inlet 3A, which is installed at the top of the pouch 1A. The washing inlet 3A has a shape of a funnel which facilitates pouring water into the pouch 1A. The washing inlet 3A also has a seal 3B which enables to seal it when not in use. In addition, the washing inlet 3A also could have an optional air filter 3C, which is installed at an opening in walls of the washing inlet 3A. The rear flexible sheet is fused all along its perimeter 1D to the perimeter of the front flexible sheet except for the washing inlet 3A opening and the draining outlet 1B opening. The fused strip 1D is shown as well. The adhesive disk 2B has tapering 2C around its circumference.

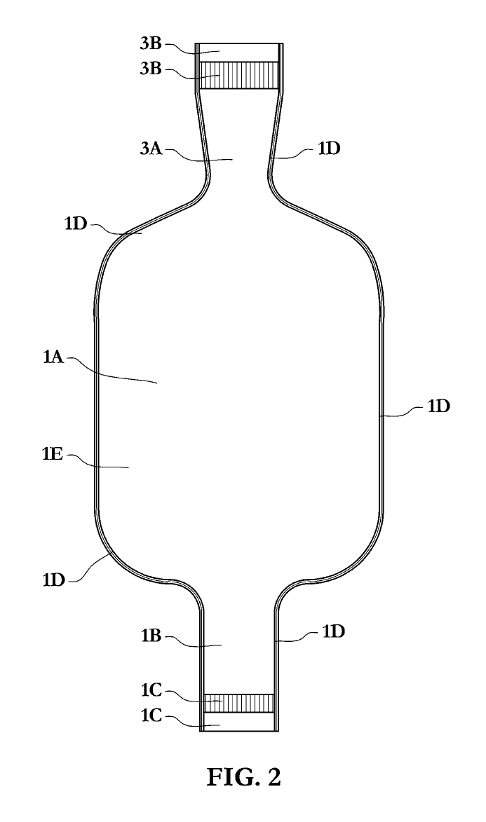

FIG. 2 illustrates the front view of an embodiment of the washable pouch 1A. The front flexible sheet 1E has two trapezoidal prostrations 3A and 1B for the washing inlet 3A and for the draining outlet 1B. The front flexible sheet 1E is fused all along its perimeter 1D to the rear flexible sheet 1F except for two short segments for the washing inlet 3A opening and the draining outlet 1B opening. The fusing strip 1D is shown as well. The washing inlet 3A has a seal 3B which enables to block all flow through the washing inlet when the seal 3B is sealed. Similarly, the draining outlet 1B has a seal 1C which enables to block all flow through the draining outlet 1B when the seal 1C is sealed.

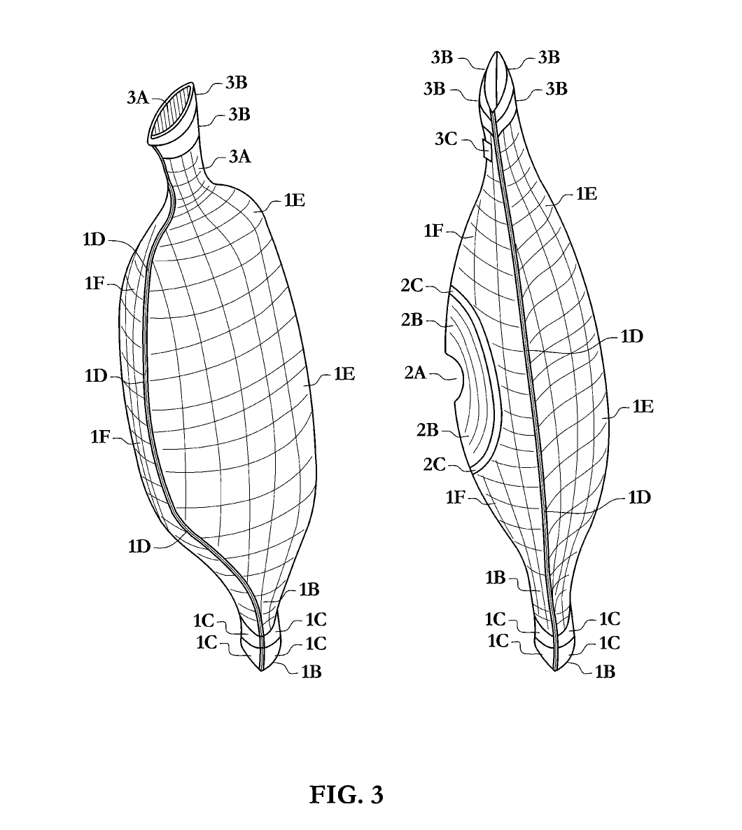

FIG. 3 shows two 3D side views of the washable pouch. In the right view both the front flexible sheet 1E and the rear flexible sheet 1F are shown. Also shown are partial side views of the abdominal inlet 2A and the adhesive ring 2B which surrounds the inlet 2A. The adhesive ring 2B is installed on the rear flexible sheet 1F. The left view shows mostly the front flexible sheet 1E. The fusing strip 1D of the front flexible sheet 1E with the rear flexible sheet 1F is drawn in both views.

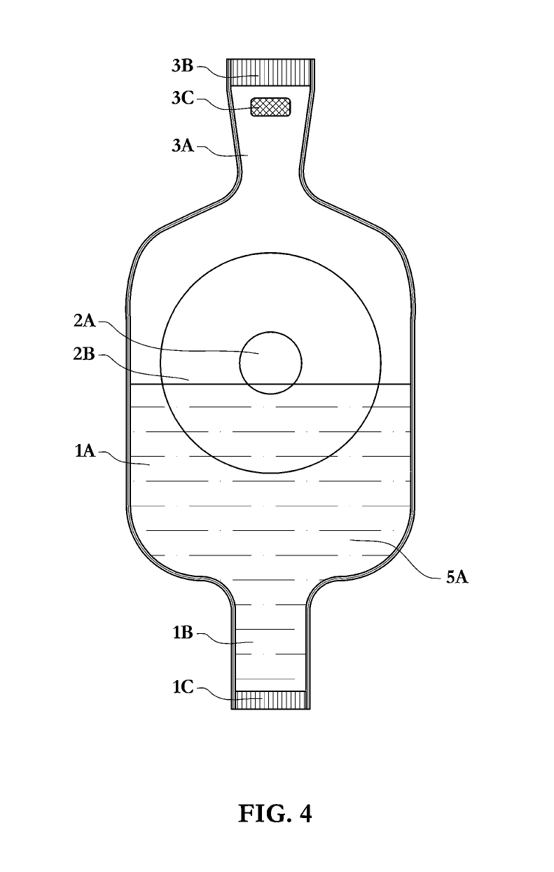

FIGS. 4-8 describe a proposed draining and washing procedure using the drainable and washable pouch 1A. FIG. 4 describes the pouch 1A in the accumulation phase when both the draining outlet 1B and the washing inlet 3A are sealed and the solid waste 5A which is excreted from the abdominal opening via the abdominal inlet 2A is accumulated in the pouch 1A.

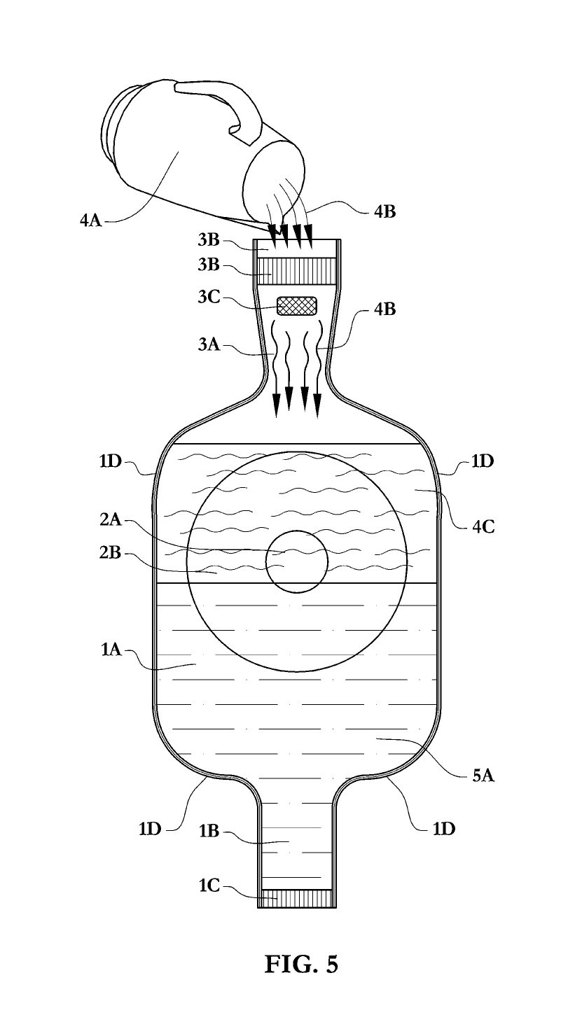

When the user decides to drain the pouch 1A, the user opens the seal 3B of the washing inlet 3A and pours water 4B into the inlet 3A. This step is illustrated in FIG. 5. The amount of water 4C which is poured from the pitcher 4A should be sufficient to create a fluent mixture with the accumulated solid waste 5A in the pouch 1A. The seal 1C of the draining outlet 1B is sealed during this phase.

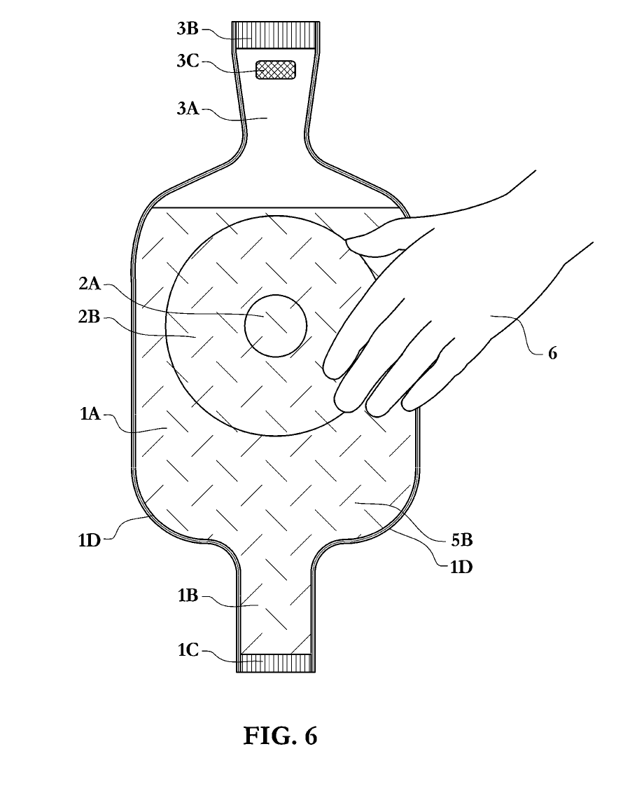

As illustrated in FIG. 6, the user then seals the washing inlet 3A and squeezes the pouch 1A with his/her hands several times until the water mixes with the solid waste and creates a fluent mixture 5B. The squeezing and mixing step is described in FIG. 6.

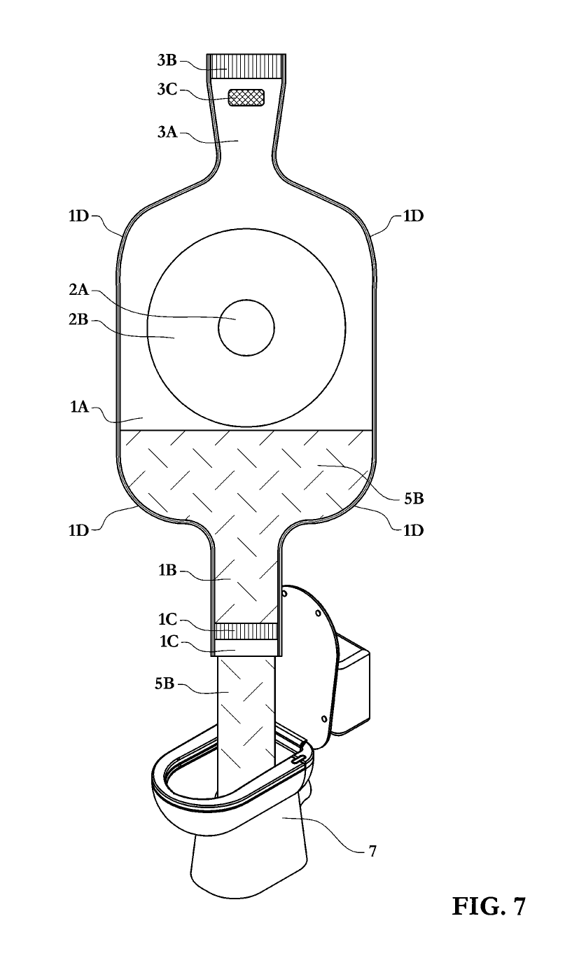

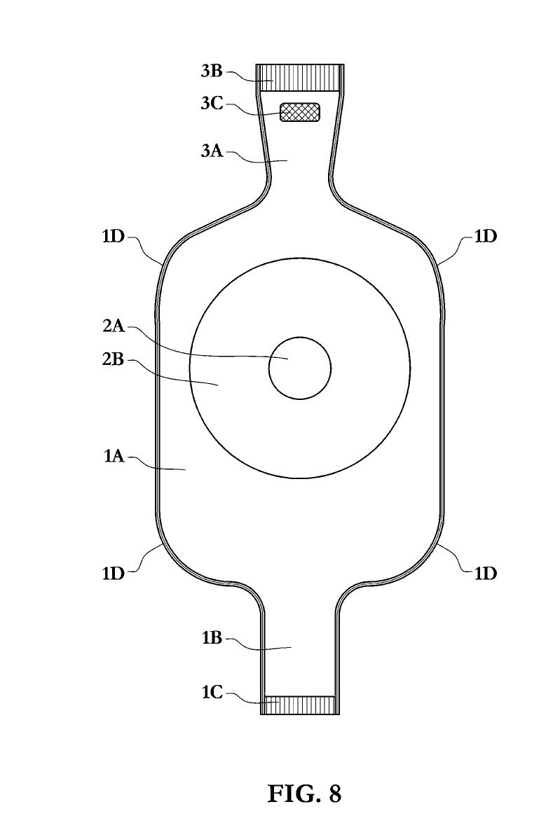

In FIG. 7, the user opens the drainage outlet seal 1C and drains the mixture 5B into the toilet bowl 7. FIG. 8 describes the state of the pouch 1A after drainage is completed. The drainage outlet 1B and the washing inlet 3A are sealed by seals 1C and 3B respectively. At this stage the pouch is ready for the next solid waste accumulation.

In our experience, almost all the solid waste 5A is drained at the first washing. If some solid waste still remains in the pouch 1A, the user can repeat the washing procedure until the pouch is completely emptied.

1. A washable pouch which enables users to accumulate solid waste in said washable pouch and also to drain and wash said washable pouch; wherein said washable pouch has an abdominal inlet; said washable pouch also has a draining outlet and a washing inlet; wherein said washable pouch, is made of a front sheet and a rear sheet; wherein both said front sheet and said rear sheet are substantially planar sheets made of flexible material; wherein said front sheet has the same two dimensional shape as said rear sheet's said two dimensional shape except for an opening for said abdominal inlet installed in said rear sheet; wherein said two dimensional shape comprises trapezoidal shape prostrations for said draining outlet and for said washing inlet; wherein said front sheet and said rear sheet are fused together at their margins all along their said two dimensional shapes' perimeter except for an opening for said draining outlet and an opening for said washing inlet.

2. Said washable pouch of claim number 1, wherein said draining outlet has a draining seal which enables said users to block any flow via said draining outlet when said draining seal is closed; wherein said draining seal allows flow via said draining outlet when said draining seal is open.

3. Said washable pouch of claim number 1, wherein said washing inlet has a washing seal which enables said users to block any flow via said washing inlet when said washing seal is closed; wherein said washing seal allows flow via said washing inlet when said washing seal is open.

4. Said washable pouch of claim number 3, wherein said washing inlet has a shape of a funnel, thereby facilitating pouring water into said washable pouch.

5. Said washable pouch of claim number 3, wherein said washing inlet is installed at the top of said washable pouch and said draining outlet is installed at the bottom of said washable pouch.

6. Said washable pouch of claim number 1, wherein said abdominal inlet is surrounded by an adhesive layer; wherein said adhesive layer is attached to said rear sheet; wherein said adhesive layer enables said users to adhere said washable pouch to said users' abdominal skin.

7. Said washable pouch of claim number 6, wherein said adhesive layer has tapered perimeter thereby reducing said users' abdominal skin irritation.

8. Said washable pouch of claim number 3, wherein an air filter is installed in an opening in the wall of said washing inlet.

9. Said washable pouch of claim number 5, wherein said draining outlet has a draining seal which enables said users to block any flow via said draining outlet when said draining seal is closed; wherein said draining seal allows flow via said draining outlet when said draining seal is open.

10. Said washable pouch of claim number 9, wherein said abdominal inlet is surrounded by an adhesive layer; wherein said adhesive layer is attached to said rear sheet; wherein said adhesive layer enables said users to adhere said washable pouch to said users' abdominal skin.

11. Said washable pouch of claim number 10, wherein said adhesive layer has tapered perimeter thereby reducing said users' abdominal skin irritation.

12. A washable pouch which enables users to accumulate solid waste in said washable pouch and also to drain and wash said washable pouch; wherein said washable pouch has an abdominal inlet; said washable pouch also has a draining outlet and a washing inlet; wherein skin of said washable pouch is made of flexible material; wherein said draining outlet has a draining seal which enables said users to block any flow via said draining outlet when said draining seal is closed; wherein said draining seal allows flow via said draining outlet when said draining seal is open; wherein said washing inlet has a washing seal which enables said users to block any flow via said washing inlet when said washing seal is closed; wherein said washing seal allows flow via said washing inlet when said washing seal is open.

13. Said washable pouch of claim number 12, wherein said washing inlet has a shape of a funnel, thereby facilitating pouring water into said washable pouch.

14. Said washable pouch of claim number 12, wherein said abdominal inlet is surrounded by an adhesive layer; wherein said adhesive layer is attached to said skin of said washable pouch; wherein said adhesive layer enables said users to adhere said washable pouch to said users' abdominal skin.

15. Said washable pouch of claim number 14, wherein said adhesive layer has tapered perimeter thereby reducing said users' abdominal skin irritation.

16. Said washable pouch of claim number 12, wherein an air filter is installed in an opening in the wall of said washing inlet.

17. Said washable pouch of claim number 12, wherein said washing inlet is installed at the top of said washable pouch and the draining outlet is installed at the bottom of said washable pouch.

18. Said washable pouch of claim number 17, wherein said washing inlet has a shape of a funnel, thereby facilitating pouring water into said washable pouch.

19. Said washable pouch of claim number 18, wherein an air filter is installed in an opening in the wall of said washing inlet.
